(19) United States
(12) Patent Application Publication
(10) Pub. No.: US 2013/0252197 A1
(43) Pub. Date: Sep. 26, 2013

(54) DENTAL RETRACTOR TOOL

(71) Applicant: Wesley Harden, Greenville, SC (US)

(72) Inventor: Wesley Harden, Greenville, SC (US)

(21) Appl. No.: 13/430,027

(22) Filed: Mar. 26, 2012

(51) Int. Cl. A61C 3/00; A61B 1/247

(52) U.S. Cl. 433/31; 433/140

(57) ABSTRACT

A dental retractor tool and method for retracting the tongue and/or cheek away from the teeth in an oral cavity is provided. The dental retractor has a shape and configuration that allows for retraction of both the tongue and cheek simultaneously while allowing a dental professional to maintain ergonomically improved posture. The dental retractor can include a neck having a U-shaped bend adapted to fit around a distal portion of a patient's tooth. The dental retractor can be located opposite another dental tool, such as a dental examination mirror, to eliminate the need for excessive instruments during a dental procedure.

The present disclosure relates generally to dental tools, and more particularly to a dental tool for retracting the cheek and/or tongue from a patient's teeth in an oral cavity.

### BACKGROUND

Dental retractor tools can be used during dental procedures to create space in an oral cavity for a dental professional to gain improved access to one or more of a patient's teeth. For example, a dental professional may use a dental retractor tool to move a tongue out of proximity to a patient's teeth so that the dental professional has adequate room to perform a dental procedure.

Certain retractor tools are configured to pull the tongue out of the way of the patient's teeth. An exemplary “pull type” retractor tool is configured with an L-shaped design that allows a dental professional to pull the tongue away from the patient's teeth. These dental retractor tools are suitable for most dental procedures, but can become cumbersome when the dental professional needs to gain access to certain teeth.

For example, a right handed dental professional can have difficulty maintaining ergonomically ideal posture when using a pull type retractor tool to gain access to a patient's teeth in the lower left quadrant. Similarly, a left-handed dental professional can have difficulty maintaining ergonomically ideal posture when using a pull type retractor to gain access to a patient's teeth in the lower right quadrant. This is primarily because the retractor tool is held in the dental professional's non-dominant hand so that the dental professional can perform dental procedures with the professional's dominant hand. Pulling a patient's tongue in a direction opposite the dental professional's non-dominant hand can lead to awkward hand positions, leading to fatigue and reduced ability to perform certain dental procedures.

Tongue retractor tools that are configured to push the tongue away from a patient's teeth are also known. While these push type retractor tools may be easier to use when accessing certain portions of the oral cavity, such as the lower left quadrant by a right-handed dental professional, these retractor tools typically include various members or portions that might block or impede access to certain teeth in the oral cavity. For instance, the handle or neck of a push type retractor tool may block or otherwise impede access to certain teeth in the oral cavity.

Thus, a need exists for an improved dental retractor tool that can be used to retract a patient's tongue and cheek away from the patient's teeth. A tool that would allow a dental professional to maintain ergonomically ideal posture while at the same time providing increased access to a patient's teeth would be particularly useful.

### SUMMARY

Aspects and advantages of the invention will be set forth in part in the following description, or may be obvious from the description, or may be learned through practice of the invention.

One exemplary aspect of the present disclosure is directed to a dental retractor. The dental retractor includes a handle having first and second end portions. The retractor further includes a neck coupled to the first end portion of the hand. The neck defines a substantially U-shaped bend having a first leg extending in a first direction and a second leg extending in a second direction. The retractor tool further includes a retractor member extending from the neck. The retractor member has a length and a width, the length of the retractor tool being longer than the width. The retractor member extends from the second leg of the neck such that the length of the retractor member extends substantially in the second direction defined by the second leg.

Another exemplary aspect of the present disclosure is directed to a method for retracting the tongue and cheek from the teeth in an oral cavity. The method includes placing a retractor tool in the oral cavity. The retractor tool includes a neck defining a U-shaped bend and a retractor member extending from the U-shaped bend. The method further includes positioning the U-shaped bend around the distal surface of a tooth and pushing the tongue away from the teeth with the retractor member.

Yet another exemplary aspect of the present disclosure is directed to a dental retractor for retracting the tongue and cheek from teeth in an oral cavity. The dental retractor includes a handle having first and second end portions. The dental retractor further includes a neck extending from the end portion having a shape adapted to fit around a distal surface of a tooth. The dental retractor further includes a retractor member extending from the neck. The retractor member has a shape adapted to press the tongue away from the teeth.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 depicts a perspective view of a dental retractor tool according to an exemplary embodiment of the present invention;

FIG. 2 depicts a perspective view of an exemplary dental retractor tool being used in an oral cavity according to an exemplary embodiment of the present disclosure;

FIG. 3 depicts a top-down view of an exemplary dental retractor tool being used in an oral cavity according to an exemplary embodiment of the present disclosure;

FIG. 4 depicts a front view of an exemplary dental retractor tool being used in an oral cavity according to an exemplary embodiment of the present disclosure;

FIG. 5 depicts a perspective view of an exemplary dental retractor tool according to an exemplary embodiment of the present disclosure; and

FIG. 6 depicts a flow diagram of an exemplary method according to an exemplary embodiment of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally, the present disclosure is directed to a dental retractor and method for retracting the tongue and/or cheek away from the teeth in an oral cavity. The dental retractor has a shape and configuration that allows for retraction of both the tongue and cheek simultaneously while allowing a dental professional to maintain ergonomically improved posture.

For example, a right handed dental professional can hold the dental retractor in the dental professional's non-dominant hand (left hand) and easily use the retractor to push the tongue away from a patient's teeth in the lower left quadrant. Similarly, a left-handed dental professional can hold the dental retractor in the dental professional's non-dominant hand (right hand) and easily use the retractor to push the tongue away from a patient's teeth in the lower right quadrant. This frees the dental professional's dominant hand for use in performing a dental procedure on the patient's teeth.

Moreover, because the retractor is configured to push the tongue away from the patient's teeth, the dental professional can retract the tongue away from patient's teeth while maintaining ergonomically improved posture without having to awkwardly position the professional's hands. For instance, the dental professional does not have to awkwardly position a non-dominant hand holding the retractor to pull the tongue from a direction opposite the non-dominant hand as is known with typical pull type retractors.

In addition, the dental retractor includes a neck defining a U-shaped bend that is adapted to fit around a distal portion of a patient's tooth. As used herein, a distal portion of a tooth refers to a surface of a tooth that faces towards the rear of the oral cavity or away from the mouth. The U-shaped bend facilitates in retracting the cheek away from the patient's teeth. The U-shaped bend also allows the dental professional to position the retractor such that neck of the retractor does not impede or block access to certain of the patient's teeth. In this manner, the dental retractor of the present disclosure can provide for unprecedented access by a dental professional to certain portions a patient's oral cavity, such as the lower left quadrant by a right handed dental professional or the lower right quadrant by a left handed dental professional.

According to additional aspects of the present disclosure, the dental retractor can be located opposite another dental tool, such a dental examination mirror. For instance, the dental retractor can be removably coupled, for instance through a screw attachment, to the opposite end of a typical dental examination mirror. This can eliminate the need for excessive instruments when performing a dental procedure. By including a retractor opposite a dental mirror, a secondary use of a historically single-use standard instrument that is typically held in a dental professional's non-dominant hand can be provided.

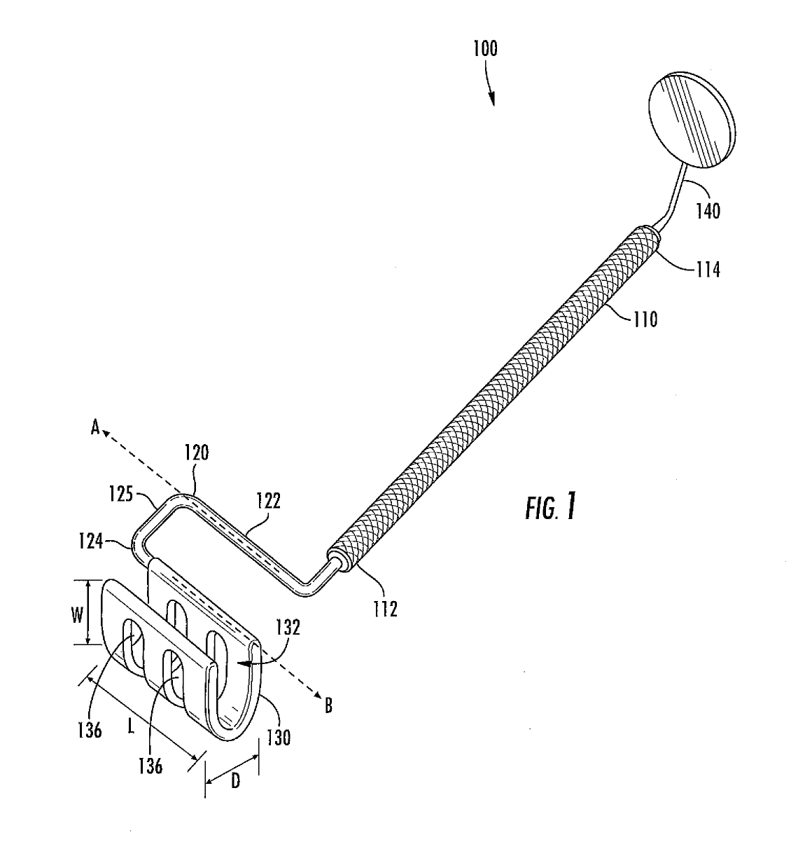

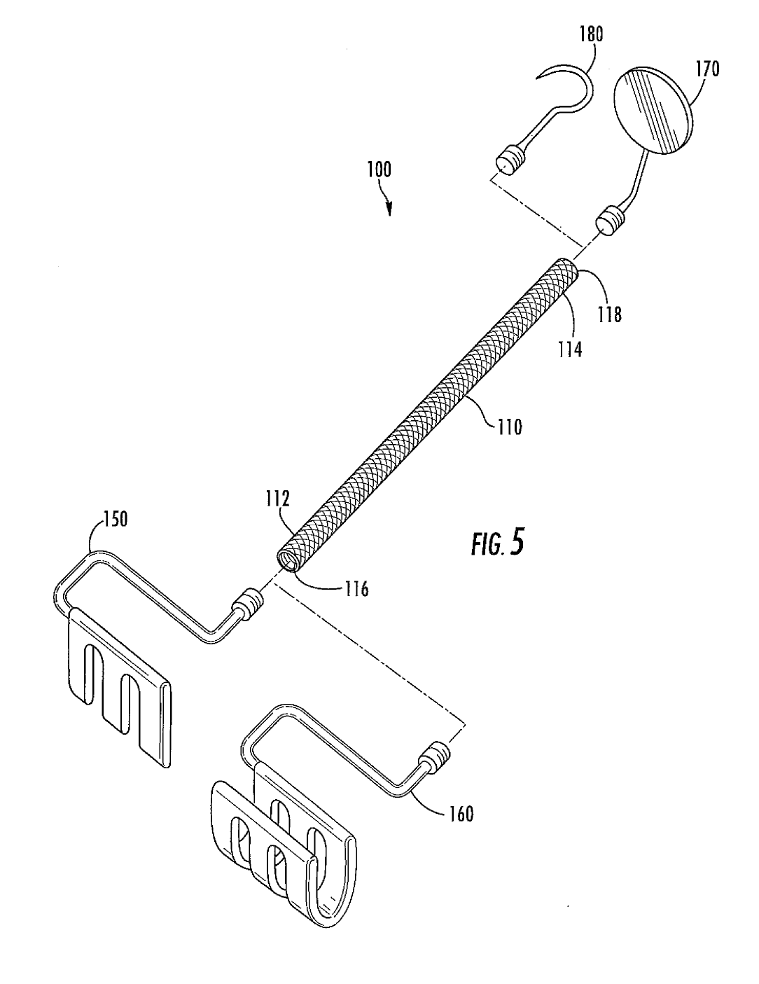

FIG. 1 illustrates an exemplary dental retractor tool 100 according to an exemplary embodiment of the present disclosure. The dental retractor tool 100 includes a handle 110, a neck 120, and a retractor member 130. The handle 110 provides a grip for a dental professional to hold the dental retractor tool 100. The handle 110 can be made from any suitable material such as stainless steel or other suitable material. The handle 110 can include a pattern on the surface of the handle 110 that facilitates gripping of the dental retractor tool 100 by a dental professional. As shown, the handle has a first end portion 112 and a second opposing end portion 114. As will be discussed in more detail with reference to FIG. 5 below, the first end portion 112 and second end portion 114 can include a screw attachment mechanism such that a variety of dental tools and retractors can be removably coupled to the handle 110 as desired.

Referring to FIG. 1, the neck 120 extends from the first end portion 112 of the handle 110. The neck 120 defines a U-shaped bend 125 that includes a first leg 122 extending in a first direction A and a second leg 124 extending in a second and opposing direction B. As will be discussed in detail below, the U-shaped bend 125 can be configured to fit around a distal portion of a patient's tooth to facilitate access to the teeth in an oral cavity. The neck 120 can be made from any suitable material. In one particular implementation, the neck 120 can be made from a bendable material that allows the dental professional to shape the neck 120 as desired for use in a particular patient's oral cavity. In this manner, the dental professional can tweak the shape of the U-shaped bend 125 and/or the first and second legs 122 and 124 to more suitably fit a particular patient.

The retractor member 130 extends from the second leg 124 of the neck 120 and is configured to press against the tongue of a patient. The retractor member 130 can be made from any material suitable for placement in a patient's oral cavity, such as a biocompatible and/or medical grade polymeric material. Those of ordinary skill in the art, using the disclosures provided herein, should understand that the present disclosure is not limited to any particular material for the retractor member 130.

The retractor member 130 has a length L, a width W, and a depth D. As shown, the length L of the retractor member 130 is longer than the width W and the depth D of the retractor member 130. As illustrated, the length L of the retractor member 130 is substantially equal to the length of the first leg 122 of the neck 120. This can allow the neck 120 to fit around a plurality of the patient's teeth as will be discussed below.

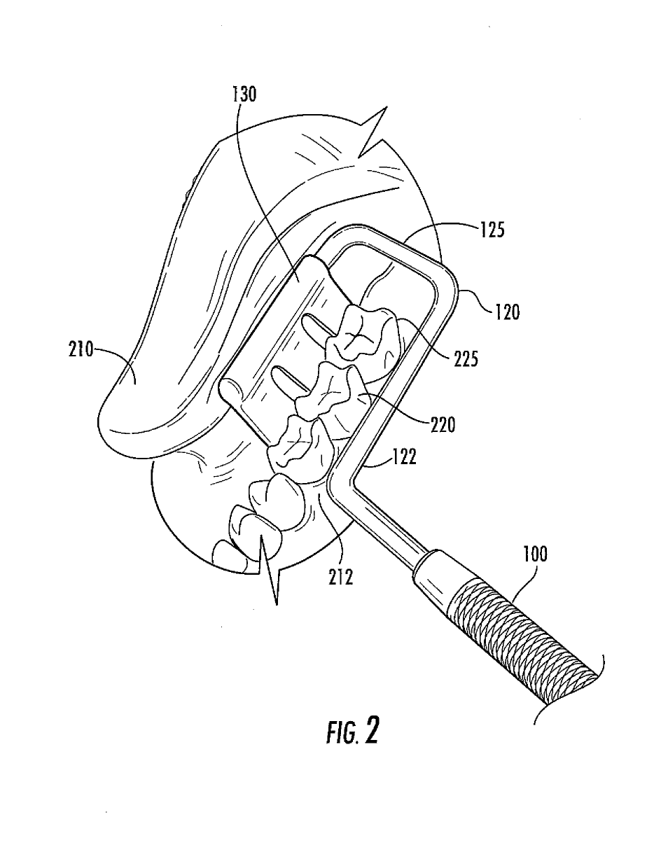

The retractor member 130 extends from the second leg 124 of the neck 120 such that the length L of the retractor member 130 extends substantially in the second direction B defined by the second leg 124 of the neck 120. In particular, the retractor member 130 extends from the second leg 124 such that the length L of the retractor member extends within 45° of the second direction B defined by the second leg 124. As will be discussed with reference to FIGS. 2-4, this allows the length L of the retractor member 130 to extend along the length of a patient's tongue when the U-shaped bend is positioned around a distal portion of a patient's tooth. As a result, the neck 120 does not impede or block access to the patient's teeth.

The retractor member 130 can be any suitable shape configured to press against a patient's tongue. For instance, the retractor member 130 can include a flat member with one or more grooves adapted to fit against a tongue in an oral cavity. The exemplary retractor member 130 depicted in FIG. 1 includes a receptacle 132. The receptacle 132 can hold cotton rolls or other materials configured to absorb saliva and other fluids during a dental procedure. The retractor member 130 can include apertures 136 to allow the saliva and other fluids to be absorbed by the cotton rolls.

As shown in FIG. 1, a dental tool 140 extends from the second end portion 114 of the handle 110. While the dental tool 140 can be any suitable dental tool, such as a dental probe, mirror or other suitable tool, it can be particularly beneficial if the dental tool 140 is a dental examination mirror as illustrated in FIG. 1. Dental examination mirrors are commonly held by the non-dominant hand of a dental professional. The retractor tool 100 of the present disclosure, provides increased access to certain quadrants of a patient's teeth (lower left quadrant for right handed dental professionals and upper right quadrant for left handed dental professionals) when used in the non-dominant hand of the professional. Thus, providing the retractor tool opposite a dental examination mirror allows for dual use of an instrument commonly held by a dental professional's non-dominant hand.

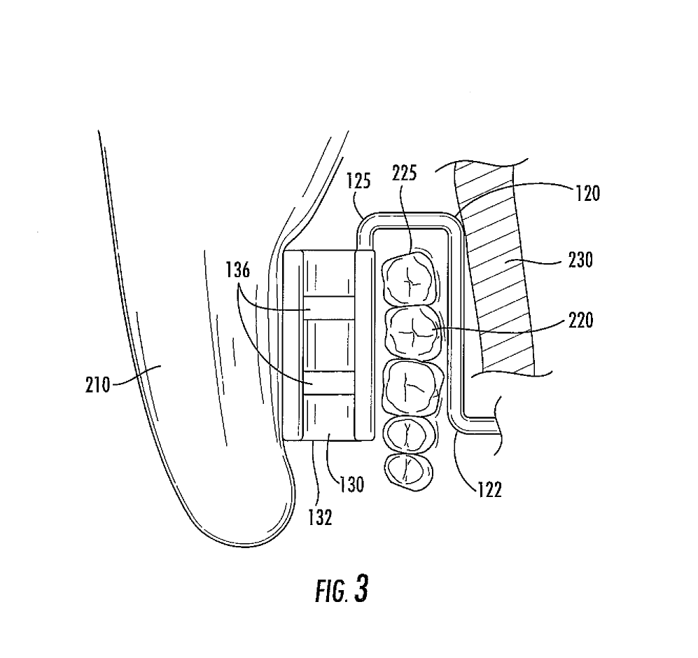

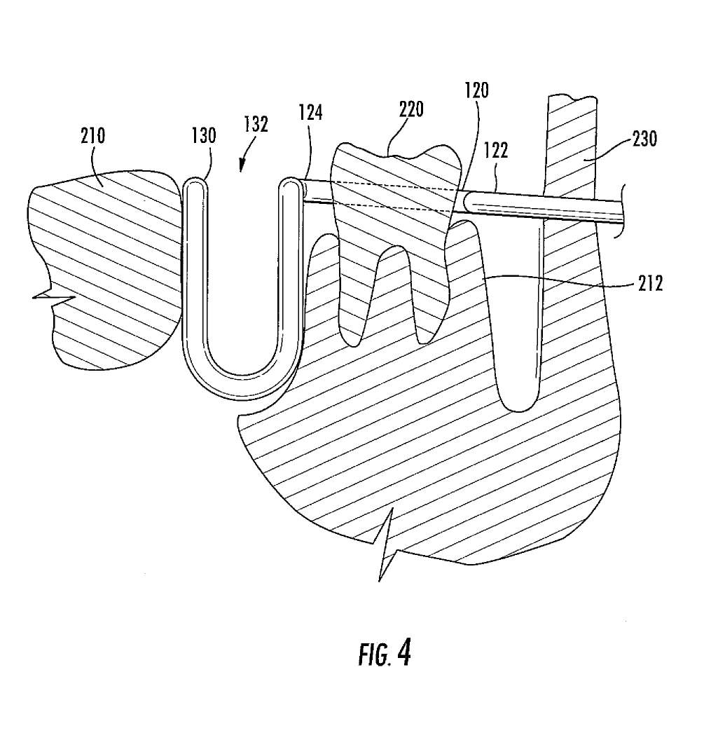

Referring now to FIGS. 2-4 the use of the exemplary retractor tool 100 to retract the tongue and/or cheek away from a patient's teeth in an oral cavity will be discussed in detail. FIG. 2 depicts a perspective view of dental tool 100 being used in a patient's oral cavity. The retractor member 130 of the retractor tool 100 in FIG. 1 is a relative flat retractor member 130 configured to push the tongue 210 of the patient away from the patient's teeth 220. As shown in FIGS. 3-4, the retractor member 130 could also have a shape that includes a receptacle 132 for receiving cotton swabs and the like.

Referring back to FIG. 2, the retractor tool 100 has been positioned such that retractor member 130 presses the tongue 210 away from the patient's teeth 220. In addition, the U-shaped bend 125 defined by the neck 120 of the retractor tool 100 has also been positioned to fit around a distal portion 225 of one of the patient's posterior teeth 220. The first leg 122 of the neck 120 runs along the gum line 212 associated with the patient's posterior teeth 220. This allows the retractor tool 100 to push the tongue 210 away from the patient's posterior teeth 220 without the neck 120 or other portions of the dental retractor tool 100 impeding or blocking access to the patient's posterior teeth 220.

A top-down view of the positioning of the retractor tool 100 in an oral cavity is depicted in FIG. 3. As shown, the retractor member 130 is positioned to push the tongue 210 away from the patient's teeth 220. The U-shaped bend 125 has been positioned to fit around a distal portion 225 of one of the patient's posterior teeth 220. As illustrated, the neck 120 fits around a plurality of the patient's posterior teeth. In addition, the first leg 122 of the neck 120 retracts the cheek 230 away from the patient's posterior teeth 220, such that the retractor tool 100 provides simultaneous retraction of the tongue 210 and cheek 230 while still providing improved access to the patient's posterior teeth 220.

A front view of the dental retractor tool 100 being used in an oral cavity is illustrated in FIG. 4. As shown, the retractor member 130 has pushed the tongue 210 away from the patient's teeth 220. The neck 120 has been positioned to fit around a distal portion of the patient's tooth. The first leg 122 retracts the cheek 230 away from the patient's teeth 220. The first leg 122 has also been positioned along the gum line 212 associated with the patient's teeth 220 so that the first leg does not impede access to the patient's teeth 220. To facilitate the positioning of the first leg 122 adjacent the patient's gum line 212, the first leg 122 can be offset from the second leg 124 of the neck 120 such that the first leg 122 is lower than the second leg 124. This further enhances the ability of the retractor tool 100 to provide increased access to the patient's teeth.

FIG. 5 depicts the dental retractor tool 100 with various components being removably coupled to the handle 110. In particular, the handle 110 can include a screw attachment 116 at a first end portion 112 of the handle 110 and a screw attachment 118 at a second end portion 114. Various retractor heads according to exemplary aspects of the present disclosure can be removably coupled to the first end portion 112 of the handle 110 through screw attachment 116. For instance, a first retractor head 150 and a second retractor head 160 can be alternately coupled to the first end portion 112 of the handle 110 as desired.

A variety of other dental tools can be removably coupled to the second end portion 114 of the handle 110 through screw attachment 118. For instance, a dental examination mirror 170, a dental probe 180, or another suitable dental tool can be removably coupled to the second end portion 114 of the handle 110 as desired. As a result, the dental retractor tool 100 can be readily adapted to perform a variety of dental functions and eliminate the need for excessive instruments during a dental procedure.

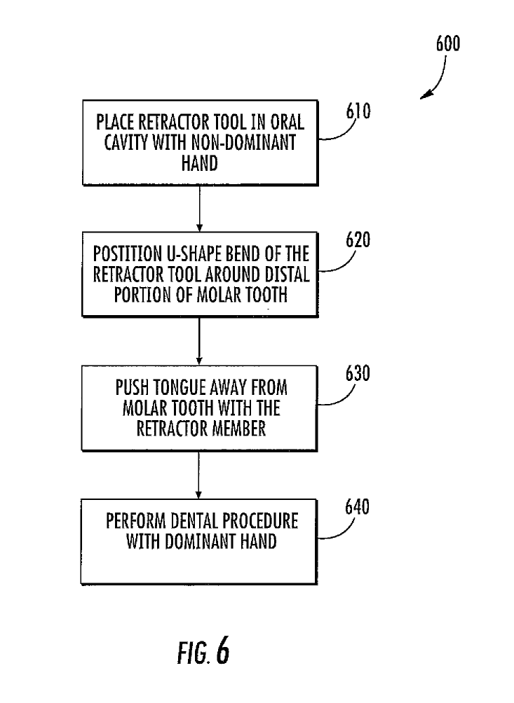

FIG. 6 depicts a flow diagram of an exemplary method (600) according to an exemplary embodiment of the present disclosure. The present method (600) will be discussed with reference to the exemplary dental retractor tool 100 illustrated in FIGS. 1-5. However, the method (600) can be performed with any suitable retractor tool falling within the scope of the present disclosure. In addition, although FIG. 6 depicts steps performed in a particular order for purposes of illustration and discussion, the methods discussed herein are not limited to any particular order or arrangement. One skilled in the art, using the disclosures provided herein, will appreciate that various steps of the methods can be omitted, rearranged, combined and/or adapted in various ways.

At (610), a dental professional or other user places the retractor tool 100 in a patient's oral cavity with a non-dominant hand. As illustrated above with respect to FIG. 1, the retractor tool 100 can include a neck 120 defining a U-shaped bend 125 having a first leg 122 extending in a first direction A and a second leg 124 extending in a second and opposing direction B. The retractor tool 100 can further include a retractor member 130 extending from the second leg 124 of the U-shaped bend 125.

At (620), the dental professional or other user positions the U-shaped bend 125 of the neck 120 around a distal portion of a patient's tooth. In particular implementations, the U-shaped bend 125 can be positioned around a plurality of teeth and the first leg 122 can be positioned adjacent the gum line associated with the patient's teeth. By positioning the U-shaped bend 125 around the distal portion of the patient's tooth, the neck 120 of the retractor tool 100 does not impede or block access to the patient's teeth.

At (630), the method includes pushing the tongue away from the patient's teeth with the retractor member 130. By pressing the tongue away from the teeth, the dental professional can more easily maintain ergonomically improved posture during the dental procedure. For instance, a right-handed dental professional can gain improved access to a patient's teeth in the lower left quadrant. Similarly, a left-handed dental professional can gain improved access to a patient's teeth in the lower right quadrant. This frees up the dental professional's dominant hand so that the dental professional can perform a dental procedure on the patient using the professional's dominant hand as shown at (640).

The dental retractor tool and method of the present disclosure has been discussed with reference to retracting the tongue and cheek away from a patient's teeth in a particular quadrant of the patient's oral cavity, such as the lower left quadrant for right-handed dental professionals and the lower right quadrant for left-handed dental professionals. Those of ordinary skill in the art, using the disclosures provided herein, should understand that the the dental tool of the present disclosure can also be modified for use in other quadrants of the oral cavity.

For instance, the retractor tool can be modified to include a first retractor portion (such as a retractor member or other portion) having a shape configured to pull the tongue away from the patient's teeth as well as an additional second retractor portion for pushing the cheek away from the patient's teeth. The first retractor portion and the second retractor portion can be connected with a neck having a U-shaped bend adapted to fit around a distal portion of a patient's tooth. This modified dental retractor tool could be suitable for use with the lower right quadrant for right-handed dental professionals and the lower left quadrant for left-handed dental professionals. In this manner, the dental tool of the present disclosure can be adapted for use in a variety of different portions of a patient's oral cavity. Other modifications can be made to the dental retractor tool to adapt the dental retractor tool for use in various quadrants of a patient's oral cavity while allowing a dental professional to maintain ergonomically improved posture.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A hand held dental retractor, comprising:
a handle having first and second end portions;
a neck coupled to the first end portion of said handle, the neck defining a U-shaped bend having a first leg extending in a first direction and a second leg extending in a second direction, the second direction being opposite to the first direction, the second leg of the neck having a length that is less than a length of the first leg of the neck; and
a retractor member extending from said neck, said retractor member having a length and a width, the length of said retractor member being longer than the width, said retractor member comprising a receptacle having a U-shaped cross-section, the retractor member further comprising one or more apertures extending across at least a portion of the receptacle, the one or more apertures having a U-shaped cross-section substantially coincident with the U-shaped cross-section of the retractor member;
wherein said retractor member extends from the second leg of said neck such that the length of said retractor member extends substantially in the second direction defined by the second leg.

2. The dental retractor of claim 1, wherein the U-shaped bend has a shape adapted to fit around a distal surface of a tooth.

3. The dental retractor of claim 2, wherein the neck has a shape adapted to fit around a plurality of teeth.

4. The dental retractor of claim 1, wherein the first leg has a shape adapted to retract a cheek away from a tooth and the retractor member has a shape adapted to press a tongue away from the tooth while the U-shaped bend fits around a distal surface of the tooth.

5. The dental retractor of claim 1, wherein the first leg of the U-shaped bend is offset from the second leg of the U-shaped bend.

6. (canceled)

7. (canceled)

8. The dental retractor of claim 1, wherein said dental retractor comprises a dental tool extending from the second end portion of said handle.

9. The dental retractor of claim 1, wherein said dental tool comprises a mirror.

10. The dental retractor of claim 1, wherein said dental tool is removably coupled to the second end portion of said handle.

11. The dental retractor of claim 1, wherein said neck and retractor member are removably coupled to said handle.

12. The dental retractor of claim 1, wherein said neck and retractor member are removably coupled to said handle with a screw attachment.

13. A method for retracting the tongue and cheek from the teeth in an oral cavity, comprising:
holding a retractor tool in a non-dominant hand;
placing the retractor tool in the oral cavity with the non-dominant hand, the retractor tool comprising a neck defining a U-shaped bend and a retractor member extending from the U-shaped bend, the neck comprising a first leg and a second leg, wherein the first leg extends in a first direction and the second leg extends in a second direction, the first direction being parallel to the second direction, the second leg having a length that is less than a length of the first leg;
positioning and holding with the non-dominant hand the U-shaped bend around the a distal surface of a tooth such that the first leg of the U-shaped bend is positioned along a gum line associated with the teeth; and
pushing the tongue away from the teeth with the retractor member.

14. The method of claim 13, wherein positioning the U-shaped bend around the distal surface of a tooth comprises positioning the U-shaped bend around a plurality of teeth.

15. (canceled)

16. A dental retractor for retracting the tongue and cheek from teeth in an oral cavity, comprising:
a handle having first and second end portions;
a neck extending from the first end portion, the neck having a shape adapted to fit around a distal surface of a tooth, the neck defining a U-shaped bend having a first leg extending in a first direction and a second leg extending in a second direction, the second direction being opposite to the first direction, the first direction being parallel to the second direction, the second leg of the neck having a length that is less than a length of the first leg of the neck; and
a retractor member extending from the neck, the retractor member having a shape adapted to press the tongue away from the teeth.

17. The dental retractor of claim 16, wherein the neck has a shape adapted to fit around a plurality of teeth.

18. The dental retractor of claim 16, wherein the neck has a shape adapted to retract the cheek away from the teeth.

19. The dental retractor of claim 16, wherein the dental retractor further comprises a dental tool coupled to the second end portion of the dental retractor.

20. The dental retractor of claim 19, wherein the dental tool comprises a mirror.

21. The dental retractor of claim 1, wherein the first leg is connected to the second leg only through the U-shaped bend.
